(19) United States
(12) Patent Application Publication
(10) Pub. No.: US 2003/0148278 A1
(43) Pub. Date: Aug. 7, 2003

(54) Test kit and method for quantitatively detecting genetically modified DNA in foodstuff by means of fluorescence-coupled PCR

(75) Inventors: Frank-Roman Lauter, Luneburg (DE); Lutz Grohmann, Berlin (DE); Roger Petersen, Berlin (DE)

Correspondence Address: BRUCE LONDA, NORRIS, MCLAUGHLIN & MARCUS, P.A., 220 EAST 42ND STREET, 30TH FLOOR, NEW YORK, NY, 10017, US

(73) Assignee: Bioinside GmbH, Teltow (DE)

(21) Appl. No.: 09/922,449

(22) Filed: Aug. 3, 2001

Related U.S. Application Data
(63) Continuation-in-part of application No. PCT/EP00/00935, filed on Feb. 7, 2000.

(30) Foreign Application Priority Data
Feb. 8, 1999 (DE) 199 06 169.6

(51) Int. Cl. C12Q 1/68; C12P 19/34

(52) U.S. Cl. 435/6; 435/91.2

(57) ABSTRACT

The invention relates to a test kit and a specific method for qualitatively and quantitatively detecting DNA in foodstuff. The invention also relates to foodstuff products which come from genetically modified organisms (GMO), preferably from the RRS gene and the Bt-176 maize gene.

This application is a continuation-in-part of PCT/EP00/009835 filed Feb. 7, 2000.

### BACKGROUND OF THE INVENTION

The invention relates to a test kit and a method for the qualitative and quantitative detection of DNA in foods and food products derived from genetically modified organisms (GMO). Furthermore, the invention relates to the use of specific primer and probe DNA sequences in said detection.

Manufacturing of foods implies securing the quality thereof and providing information for consumers relating to the ingredients and the composition of food products.

Since September 1998, the Directive No. 1139/98 by the EU Council is in force in Europe, according to which foods including DNA from genetically modified organisms (GMO DNA) must be labelled. The same Directive recommends a lower limit of the GMO DNA quantity designed for future use, from which limit on foods must be labelled. Surveillance of this Directive with respect to detectability of GMO DNA and compliance with said limit necessitates a quantitative GMO DNA detection system operating with high sensitivity and reproducibility.

At present, GMO DNA is detected using the polymerase chain reaction (PCR) (Hupfer et al. 1997, Z. Lebensm. Unters. Forsch. 205, 442-445). For quantitative detection of GMO DNA, the method of quantitative competitive PCR is used (Studer et al. 1998, Z. Lebensm. Unters. Forsch. 207, 207-213).

In quantitative competitive PCR, the target genes are amplified in the presence of well-defined quantities of a standard DNA. The standard DNA is amplified by the same primers as the target genes. The resulting PCR amplification products for the target genes and for the standards are different in size and therefore can be separated by gel electrophoresis. Following PCR, the PCR products are stained, separated by gel electrophoresis, and the amounts of target gene amplification products and standard DNA amplification products are determined by densitometry. As standard DNA copies are added in well-defined amounts, the amount of target gene copies can be estimated by comparing the standard PCR products with the target PCR products.

While genetically modified DNA generally can be detected using PCR and the amount thereof can also be detected quantitatively using quantitative competitive PCR, these methods are poorly suited for routine analyses of products, particularly of foods. The methods are insufficiently automatizable, involve the risk of PCR product contaminations (false-positive results), and furnish results of insufficient precision.

### SUMMARY OF THE INVENTION

It was therefore the object of the invention to provide a method of detecting genetically modified DNA in foods, which method would permit highly automatizable and highly reproducible analyses free of contaminations.

According to the invention, said object is accomplished by using a fluorescence-coupled PCR and employing special primer and probe combinations in a specific process variant as illustrated schematically in FIG. 1.

Fluorescence-coupled PCR is a per se known method. Thus, U.S. Pat. No. 5,210,015 and U.S. Pat. No. 5,487,972 (TaqMan®) describe a PCR reaction involving three oligonucleotides—two primers and one fluorogenic probe. The probe consists of an oligonucleotide, the 5′ end of which is labelled with a reporter fluorescent dye (fluorescein derivative), while the 3′ end bears a quencher dye (rhodamine derivative) and is blocked with a phosphate residue in addition. When exciting the intact probe at a specific wavelength (488 nm) to fluoresce, the fluorescence of the reporter dye will be suppressed by a fluorescence energy transfer (FET) as a result of the spatial proximity to the quencher. During the PCR, the probe with the primers initially hybridizes to the template strand of the DNA. In the extension phase, the probe positioned between the primers contacts the Taq polymerase and is nicked by the exonuclease activity of the Taq polymerase. As a result of probe hydrolysis, the spatial proximity and thus, also the FET, between reporter and quencher is no longer present. The reporter fluorescence will increase with each PCR cycle in accordance with the accumulation of PCR product. The signal being formed is strictly sequence-specific because probe molecules not 100% bound will be displaced even before the exonuclease activity of the Taq polymerase has been activated. Ultimately, the change in fluorescence of the different dyes can be detected cycle by cycle in the sealed reaction vessel using commercial instruments such as ABIPRISM 7700 supplied by Perkin Elmer Applied Biosystems Division (PEABD).

Now, the detection according to the invention is effected by initially extracting the total DNA from the food sample according to methods per se known to those skilled in the art (cf., Zimmermann et al., 1998, Z. Lebensm. Unters. Forsch. A 207, 81-90) and—as illustrated in FIG. 1—

a) determining the amount of transgene in the total DNA by performing a PCR reaction with a transgene-specific fluorescence-labelled probe S1 and two transgene-specific primers P1 and P2 in a first reaction vessel and measuring the change in fluorescence radiation as compared to a control, a synthetic gene fragment (target IAC DNA) in addition to the primers P1 and P2 and a fluorescence-labelled probe S2 being used as internal amplification control (IAC) for transgene determination in the first reaction vessel, which fragment has two binding sites for the primers P1 and P2 and one binding site for the fluorescence-labelled probe S2 which not only differs from probe S1 in its sequence but also is labelled with a fluorescent dye other than that of probe S1;

b) selecting a reference gene and determining the amount of reference gene in the total DNA by performing a PCR reaction with a reference gene-specific fluorescence-labelled probe S3 and two reference gene-specific primers P3 and P4 in a second reaction vessel and measuring the change in fluorescence radiation as compared to a control, a synthetic gene fragment (reference IAC DNA) in addition to the primers P3 and P4 and the fluorescence-labelled probe S2 being used as internal amplification control (IAC) for reference gene determination in the second reaction vessel, which fragment has two binding sites for the primers P3 and P4 and one binding site for the fluorescence-labelled probe S2 which is identical to the fluorescence-labelled probe S2 in the target gene system, but differs from probe S3 with respect to sequence and fluorescent dye; and

ultimately, calculating the level of genetically modified DNA from the ratio of the amounts of transgene and reference gene.

In a fluorescence-coupled PCR, the amount of gene copies under investigation correlates with a measured change in fluorescence as compared to a control PCR reaction with no DNA. Thus, based on the measured change in fluorescence, it is possible to calculate the amount of sample DNA employed.

### BRIEF DESCRIPTION OF THE DRAWING

FIG. 1—Schematic Illustration of the method of detecting genetically modified DNA in foods.

### DESCRIPTION OF THE PREFERRED EMBODIMENTS

To illustrate the invention in more detail, definitions will be given hereinbelow as to how particular terms are to be understood in the meaning of the invention:

Primers

Primers are DNA oligonucleotides. Under appropriate conditions, they will hybridize only to the complementary DNA sequence of the gene fragment to be detected. They serve as starting points for the initiation of DNA synthesis by the DNA polymerase enzyme. The position of two primers within a gene determines which gene fragment can be multiplied by PCR and detected subsequently.

Probes

Probes are DNA oligonucleotides having reporter dyes coupled thereon. Under appropriate conditions, they will hybridize only to the complementary DNA sequence of the gene fragment to be detected.

Fluorescence-Coupled PCR

In a fluorescence-coupled PCR, the probes are located between the primers. In the presence of a light source inducing fluorescence radiation, a successful PCR reaction will result in a change of the probe fluorescence. The change in probe fluorescence radiation correlates with the amount of PCR products being formed and thus, with the amount of gene copies originally investigated. The fluorescence radiation can be used to calculate the amount of a gene to be detected (Heid et al. 1996, Genome Methods 6, 986-994; Wittwer et al. 1997, Bio-Techniques 22, 130-138).

Transgene

Transgenes are defined to be nucleotide sequences manipulated by human activity, e.g. by transforming them from one organism to another organism in which they are not present naturally.

Reference Gene

A reference gene is defined to be a nucleotide sequence that is present in all relevant variants of a type of organism to be detected. The reference gene serves as a reference value in the relative quantification of transgenes. The amount of reference gene and thus, the number of copies thereof, is defined to be 100% in the quantification, and the number of copies of the transgene is subsequently correlated thereto.

Target DNA

The target DNA is the nucleotide sequence which is recognized and amplified by a PCR system. The nucleotide sequences can be of various origin. The reference gene is the genomic DNA of a particular gene fragment which is present in all relevant variants of a species (e.g. soja or maize). The transgene is the genetically modified DNA of a particular DNA fragment, the existence of which DNA is to be detected. The target IAC DNAs represent synthetic DNA fragments according to the invention which, on the one hand, contain the nucleotide sequences of the specific primers and, on the other hand, contain the nucleotide sequence of the universal probe S2 of the invention.

Internal Amplification Control (IAC)

The internal amplification control controls the efficiency of a PCR reaction of a particular primer combination. It comprises the target IAC DNA of the invention or the reference IAC DNA of the invention, and the universal probe S2 of the invention.

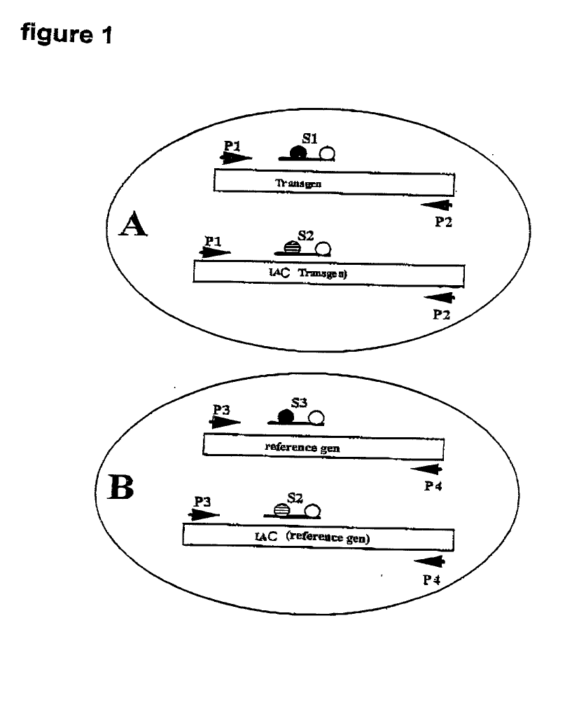

Thus, according to the invention, the following parameters are measured to allow determination of the amount of a genetically modified DNA in foods (cf., FIG. 1):

(1) The amount of transgene in the total DNA is measured using the primers P1 and P2 and the probe S1. This measurement takes place in reaction vessel (A).

(2) The amount of reference gene in the total DNA is measured using the primers P3 and P4 and the probe S3. This measurement takes place in reaction vessel (B).

The ratio of transgene to reference gene furnishes the percentage of genetically modified DNA in the DNA of a particular type of organism, the amount of reference gene reflecting the quantity of relevant DNA to be investigated.

(3) The efficiency of each PCR reaction is measured in these two reaction vessels (A) and (B) using the primers P1 to P4, the probe S2 and the target IAC DNA and reference IAC DNA according to the invention.

The specific internal amplification controls (IAC) are comprised of the inventive specific primers P1 and P2 for the transgene and P3 and P4 for the reference gene, the inventive universal probe S2, and the inventive target IAC DNA for the transgene system and the inventive reference IAC DNA for the reference gene system. The IACs permit control of the DNA quality. To this end, the PCR efficiency of both the transgene and the reference gene system is measured.

Provision of these four primer/probe systems according to the invention (cf., FIG. 1) permits control of both the DNA quantity and the quality thereof, thereby providing the necessary and sufficient preconditions for DNA quantification. According to the invention, it is possible to measure two PCR systems per reaction vessel in parallel because the probes for both systems are labelled with different fluorescent dyes. For example, the probes S1 and S3 can be labelled with the same reporter dye and S2 with a different reporter dye.

In preferred embodiments of the invention, it is possible e.g. to detect the Roundup Ready soy gene (RRS gene) and the Bt-176 maize gene as transgenes frequently occurring in foods. However, the detection method according to the invention can also be used and is excellently suited for other maize transgenes, such as the transgene in Bt-11 maize, or in the detection of the 35S CMV promoter in the form of a screening procedure in a non-specific detection of GMO DNA, or for all those transgenes introduced in the food market in future.

In a particularly preferred embodiment of the invention, the new sequences SEQ ID NO. 3 or SEQ ID NO. 3a and the new sequences SEQ ID NO. 4 or SEQ ID NO. 4a, or the variants thereof obtained by deletion, substitution or addition, which can be produced on a chemical-synthetic route, are used as primer P1 and as primer P2, respectively, in the inventive detection of the RRS gene which has the NA sequence SEQ ID NO. 1 and, according to experts' estimates, is contained in 20,000-30,000 kinds of foods. The new NA sequence SEQ ID NO. 2 or SEQ ID NO. 2a or the variants thereof obtained by deletion, substitution or addition were found to be particularly suitable as probe S1. The preparation thereof can also be performed on a chemical-synthetic route.

The lectin gene was found to be particularly suited as reference gene in the inventive detection of the RRS gene. When using lectin (cf., SEQ ID NO. 11) as reference gene, the sequences SEQ ID NO. 6 or SEQ ID NO. 6a and SEQ ID NO. 7 or variants thereof obtained by deletion, substitution or addition are used as primers P3 and P4 in a preferred embodiment of the invention. The new NA sequence SEQ ID NO. 5 or variants thereof obtained by deletion, substitution or addition were found to be particularly suited as probe S3. Like probe S1, the probe S3 is labelled at its 5′ end or its 3′ end with a reporter fluorescent dye, and with a quencher at its other end.

According to the invention, internal amplification controls are used in the transgene determination and in the reference gene determination. The NA sequences SEQ ID NO. 8 or variants thereof obtained by deletion, substitution or addition were found to be particularly suitable internal amplification controls for the transgene determination (target IAC DNA), and the NA sequence SEQ ID NO. 10 or SEQ ID NO. 10a or variants thereof obtained by deletion, substitution or addition were found to be particularly suitable internal amplification controls for the reference gene determination in the detection of the RRS gene.

In another particularly preferred embodiment of the invention, the sequence SEQ ID NO. 13 is used as primer P1 and the sequence SEQ No. 14 or variants thereof having at least 80% homology are used as primer P2 in the detection of the Bt-176 maize gene, the sequence of which is well-known and has been published in WO 93/07278. The sequence SEQ ID NO. 12 or variants thereof having at least 80% homology were found to be particularly suited as probe S1. The probe S1 is labelled with a reporter dye and a quencher as described below.

According to the invention, the invertase gene of maize, the sequence of which has been published by Xu J. et al. in Plant Physiol. July 1995, 108(3), 1293-4, is used as reference gene for detecting the Bt-176 maize gene. When using this reference gene, the sequence SEQ ID NO. 16 is used as primer P3 and the sequence SEQ ID NO. 17 or variants thereof having at least 80% homology are used as primer P4 in a preferred embodiment of the invention. In this case, the sequence SEQ ID NO. 15 or variants thereof are used as reference-specific probe S3. Likewise, the probe S3 is labelled as described below.

As internal amplification controls, the sequence SEQ ID NO. 18 and the sequence SEQ ID NO. 19 or variants thereof having at least 80% homology are used as target IAC DNA and as reference IAC DNA, respectively, in the inventive method of detecting the Bt-176 maize gene.

In a preferred embodiment, the NA sequence SEQ ID NO. 9 or variants thereof obtained by deletion, substitution or addition and having at least 80% homology are used as universal probe S2 in all the detections. At its 5′ end or its 3′ end, the probe S2 is labelled with a reporter fluorescent dye, preferably a fluorescein derivative selected from 6-carboxyfluorescein, tetrachloro-6-carboxyfluorescein, 2,7-dimethoxy-4,5-dichloro-6-carboxyfluorescein, hexachloro-6-carboxyfluorescein, which dye is different from that of probes S1 and S3, e.g. with tetrachloro-6-carboxyfluorescein. At its other sequence end, the probe is labelled with a quencher, preferably a rhodamine derivative identical to that of probes S1 and S3. 6-carboxytetramethylrhodamine was found to be particularly suitable for this purpose.

Likewise, the probes S1 and S3 are labelled at their 5′ end or 3′ end with a reporter fluorescent dye, preferably a fluorescein derivative selected from 6-carboxyfluorescein, tetrachloro-6-carboxyfluorescein, 2,7-dimethoxy-4,5-dichloro-6-carboxyfluorescein, hexachloro-6-carboxyfluorescein. In a particularly preferred embodiment, 6-carboxyfluorescein (FAM) is used as label. A rhodamine derivative, most preferably 6-carboxytetramethylrhodamine, is used as quencher at the other sequence end.

The invention is not only directed to the primer and probe sequences specifically mentioned in the specification and claims, but also to variants thereof obtained by deletion, substitution or addition, provided they have a homology of at least 80%, preferably at least 90%, to the explicitly mentioned sequences and ensure similarly good test specificity and test sensitivity as the explicitly mentioned sequences.

All of the primer and probe sequences can be prepared on a chemical-synthetic route according to methods well-known to those skilled in the art. Except for the RRS gene sequence, the lectin gene sequence, the Bt-176 gene sequence, the invertase gene sequence, and the primer sequence NO. 6, these are new sequences. The sequences are illustrated in the sequence listing which is part of this specification.

Using the highly specific and highly sensitive method according to the invention, it is possible for the first time, by virtue of the process design and the special primer and probe combinations, to specify the accuracy of the test and the GMO DNA detection limit for each test on a product.

In addition to the detection method, the invention is also directed to a corresponding test kit used to detect genetically modified DNA in foods, particularly the RRS gene and the Bt-176 maize gene. Preferably, the described sequences of the probes, primers and internal amplification controls are used. In a particularly preferred embodiment, sequence SEQ ID NO. 9 is used as universal probe S2.

According to the invention, the test kit includes a transgene-specific, fluorescence-labelled probe S1 and two transgene-specific primers P1 and P2, a reference gene-specific, fluorescence-labelled probe S3, and two reference gene-specific primers P3 and P4, as well as a fluorescence-labelled probe S2 as internal amplification control, which probe differs from probes S1 and S3 both in its sequence and its fluorescence-labelling, a synthetic gene fragment (target IAC DNA) having two binding sites for the primers P1 and P2 and one binding site for the probe S2, and a synthetic gene fragment (reference IAC DNA) having two binding sites for the primers P3 and P4 and one binding site for the probe S2.

It has been found that the detection of GMO DNA can also be performed with high precision by fluorescence-coupled PCR using hybridization probes and the process design according to the invention. To this end, two fluorogenic probes each time are used instead of the single probe S1 and the single probe S3. The 3′ end of one probe is labelled with a donor dye (fluorescein derivative), while the 5′ end of the neighboring probe is labelled with an acceptor dye (e.g. Cy5, Light Cycler Red 640 or Light Cycler Red 710). The nucleotide sequences of the probes are selected such that probes in direct neighborhood will hybridize to the target DNA.

This results in a direct spatial proximity of donor dye and acceptor dye. When exciting the pair of probes at a specific wavelength to fluoresce, the fluorescence of the donor dye will be transferred to the acceptor dye. Donor and acceptor dyes are selected to emit at different wavelengths. An increase of acceptor dye emission can only be measured when the excitation energy of the donor dye has been transferred to the acceptor dye after hybridization of both probes to the target DNA. The more PCR products formed, the more pairs of probes can hybridize, and the higher the increase of emission radiation of the acceptor dye. As in the TaqMan technology, the amount of fluorescence radiation generated is proportional to the amount of PCR products formed and thus, to the amount of starting DNA under investigation.

With reference to the embodiments, the invention will be illustrated in more detail below.

Embodiment 1:

Detection of the RRS Gene in Soy Flour SB2 From the FLUKA Company (Deisenhofen, Germany)

Using 200 mg soy flour each time, total DNA was isolated using the CTAB method (Zimmermann et al. 1998, Z. Lebensm. Unters. Forsch. A 207, 81-90). The yield was about 500 μg of DNA per 200 mg of soy flour. The isolated DNA was analyzed in PCR reactions in the presence of the transgene system or the reference gene system. To this end, the components and procedures were used as described below. All components, including primers and probes, were purchased from the company PE Applied Biosystems Division, Weiterstadt, Germany. Preparation of the TaqMan PCR reaction mixtures, performing the PCR reaction, and operating the PCR hot stage and the fluorescence detector (PEABD Model 7700 or Model LS50B) were effected according to the instructions of the instrument manufacturer (User's Manual, ABI Prism 7700 Sequence Detection System, PE Applied Biosystems Division, Foster City, USA 1997, and User's Manual, PE ABD LS50B).

For PCR amplification of the isolated DNA, the following components were mixed in a PCR reaction vessel for the transgene system and for the reference gene system, respectively (PEABD Order No. N80105080). PCR reactions of 50 μl volume per reaction were performed.

| | | |
|---|---|---|
| Reaction component | Volume (μl) | Final concentration |

Transgene system (Roundup Ready system/RRS)

| | | |
|---|---|---|
| DNA | 5.000 | |
| H₂O | 16.750 | |
| 10 × buffer A | 5.000 | 1 x |
| 25 mM MgCl₂ | 7.000 | 3.5 mM |
| 15 μM RRS primer P1 | 2.000 | 600 nM |
| 15 μM RRS primer P2 | 2.000 | 600 nM |
| 4 μM RRS probe S1 | 2.000 | 160 nM |
| 2 μM IAC probe S2 | 3.000 | 120 nM |
| dATP | 0.400 | 200 μM |
| dCTP | 0.400 | 200 μM |
| dGTP | 0.400 | 200 μM |
| dUTP | 0.800 | 400 μM |
| 5 U/μl AmpliTag Gold | 0.250 | 0.025 U/μl |
| Polymerase | | |
| RRS IAC target DNA | 5.000 | 100 gene copies |
| (109 dil plasmid) | | |
| Total volume | 50.000 | |

Reference gene system (lectin system)

| | | |
|---|---|---|
| DNA | 5.000 | |
| H₂O | 14.750 | |
| 10 × buffer A | 5.000 | 1 x |
| 25 mM MgCl₂ | 11.000 | 5.5 mM |
| 15 μM lectin primer P3 | 2.000 | 600 nM |
| 15 μM lectin primer P4 | 2.000 | 600 nM |
| 3 μM lectin probe S3 | 2.000 | 120 nM |
| 2 μM IAC probe S2 | 3.000 | 120 nM |
| dATP | 0.400 | 200 μM |
| dCTP | 0.400 | 200 μM |
| dGTP | 0.400 | 200 μM |
| dUTP | 0.800 | 400 μM |
| 5 U/μl AmpliTaq Gold | 0.250 | 0.025 U/μl |
| Polymerase | | |
| Lectin IAC target DNA | 3.000 | 100 gene copies |
| (109 dil plasmid) | | |
| Total volume | 50.000 | |

The following pipetting regimen was used in the quantification of soy flour samples (reaction volume per reaction: 50 μl)

(i) NTC

Two no template control (NTC) reactions per system (transgene and reference gene) were prepared each time. Except for the IACs, these reactions do not include any DNA and will not yield any reporter fluorescence radiation of the S1 and S3 probes in a medium free of contaminations. These negative controls ensure the absence of false-positive results.

(ii) Standard Straight Lines

Each time, 3 reactions of 4 different dilutions of 100% transgenic Roundup Ready DNA were prepared. 10 ng, 5 ng, 0.5 ng, and 0.25 ng of DNA was used in the dilutions. These 12 reactions were prepared both for the transgene and the reference gene system according to the above-mentioned regimen. Thus, 24 PCR reactions were prepared in order to obtain 2 standard straight lines—one for the transgene and one for the reference gene system.

(iii) Sample Examination

Each time, 2 reactions of 2 different dilutions of DNA were prepared, which had been extracted from soy flour having a 2% content of Roundup Ready soy. 10 ng and 5 ng of DNA was used in the dilutions. These 4 reactions were prepared both for the transgene and the reference gene system according to the above-mentioned regimen. Thus, 8 PCR reactions were prepared to examine the sample DNA.

After completion of the pipetting operations to perform the PCR reaction, the reaction vessels, totalling 36, were transferred into the hot stage of the PEABD Model 7700 sequence detector. The following temperature regimen was adjusted in the PCR amplification:

Temperature conditions

| | | |
|---|---|---|
| 10 min | 95° C. | hold |
| 15 s | 95° C. | 45 |
| 60 s | 60° C. | cycle |
| 2 min | 25° C. | hold |

The PEABD Model 7700 sequence detector was started according to the instructions of the manufacturer. The PCR conditions had been optimized by varying the primer and probe designs, the primer and probe concentrations, the MgCl₂ concentration, and the oligonucleotide annealing temperature.

During the PCR reaction, the change in fluorescence radiation was measured by the fluorescence detector of the PEABD Model 7700 sequence detector.

As a measure for the amount of PCR products formed and thus, of target gene copies employed, the so called threshold cycle value or Ct value is used in the TaqMan technology.

Ct value: The hydrolysis of the fluorescence probe occurring during the TaqMan PCR results in an increase of the reporter fluorescence radiation from one PCR cycle to the next. The number of cycles where the reporter fluorescence radiation is higher than the background radiation (NTC=no template control) of the system for the first time is referred to as “threshold cycle” (Heid et al. 1996, Genome Methods 6, 986-994). Background radiation (NTC) is the reporter fluorescence radiation in PCR control reactions wherein no template DNA was used.

Both the amount of reporter radiation emitted and the Ct value are proportional to the amount of target gene copies employed. The more gene copies employed, the lower the resulting Ct value. In a PCR system with 100% efficiency, the Ct value will decrease by one cycle each time the starting number of gene copies is doubled. In a PCR reaction comprising e.g. 45 cycles wherein no PCR product is formed, the Ct value will be 45 by definition. Prior to beginning the quantification, the Ct values and the curve profile of the change in radiation of the IAC probe S2 is controlled for both systems. In the absence of PCR inhibitors, the Ct value and curve profile of the change in radiation of S2 in the tested samples will correspond to those of S2 in the NTC. In such a situation, the presence of sample DNA will not affect the efficiency of the PCR and therefore, the Ct values obtained can be used in the quantification calculations. Thus, if the IAC PCR reactions are not affected by the presence of sample DNA, the quantification is started. In case the IAC PCR reactions are affected by the sample DNA, the PCR reactions must be repeated using more carefully purified sample DNA.

Using the standard straight lines, the Ct values obtained for IAC-positive values were converted into the relative numbers of gene copies. The ratio of the relative number of transgene copies to the number of reference gene copies furnishes the percentage of GMO DNA and total soy DNA of the tested sample. Indeed, the quantification of the transgene DNA versus reference gene DNA for the soy flour sample tested in this Example and having a percentage of 2% Roundup Ready soy flour FLUKA SB 2 No. 85478 furnished 2.00%±0.30%. The error evaluation to determine the precision range was performed according to User Bulletin #2, ABI Prism 7700 Sequence Detection System, 1997, p. 34.

Thus, the system described herein fully complies with the intended function.

Embodiment 2:

Detection of the Bt-176 Maize Gene in MZ 2 Maize Flour From FLUKA Company

In an analogous fashion as in Embodiment 1 and using the sequences ID NOs. 12-19 as described above, the percentage of Bt-176 maize DNA in 200 mg of maize flour was determined. The quantification of the transgene DNA versus reference gene DNA for the maize flour sample tested in this Example and having a percentage of 2% FLUKA maize flour MZ 2 No. 63198 furnished 1.91+0.25%. The error evaluation was performed as in Example 1.

1. A method of quantitatively detecting genetically modified DNA (transgene) in foodstuffs,
characterized in that
the detection is performed using fluorescence-coupled PCR, said method involving extracting the total DNA from the food sample, and
a) determining the amount of transgene in the total DNA by performing a PCR reaction with a transgene-specific fluorescence-labelled probe S1 and two transgene-specific primers P1 and P2 in a first reaction vessel and measuring the change in fluorescence radiation as compared to a control, a synthetic gene fragment (target IAC DNA) in addition to the primers P1 and P2 and a fluorescence-labelled probe S2 being used as internal amplification control (IAC) for transgene determination in the first reaction vessel, which fragment has two binding sites for the primers P1 and P2 and one binding site for the fluorescence-labelled probe S2 which not only differs from probe S1 in its sequence but also is labelled with a fluorescent dye other than that of probe S1;
b) selecting a reference gene and determining the amount of reference gene in the total DNA by performing a PCR reaction with a reference gene-specific fluorescence-labelled probe S3 and two reference gene-specific primers P3 and P4 in a second reaction vessel and measuring the change in fluorescence radiation as compared to a control, a synthetic gene fragment (reference IAC DNA) in addition to the primers P3 and P4 and the fluorescence-labelled probe S2 being used as internal amplification control (IAC) for reference gene determination in the second reaction vessel, which fragment has two binding sites for the primers P3 and P4 and one binding site for the fluorescence-labelled probe S2 which is identical to the fluorescence-labelled probe S2 in the target gene system, but differs from probe S3 with respect to sequence and fluorescent dye; and
ultimately, calculating the level of genetically modified DNA from the ratio of the amounts of transgene and reference gene.

2. The method according to claim 1, wherein the Roundup Ready soy gene (RRS gene) is detected as transgene.

3. The method according to claim 1, wherein the nucleic acid sequence SEQ ID NO. 2 or SEQ ID NO. 2a or variants thereof obtained by deletion, substitution or addition and having at least 80% homology are used as probe S1.

4. The method according to claim 1, wherein the nucleic acid sequence SEQ ID NO. 3 or SEQ ID NO. 3a and the nucleic acid sequence SEQ ID NO. 4 or SEQ ID NO. 4a or the variants thereof obtained by deletion, substitution or addition and having at least 80% homology are used as primer P1 and primer P2, respectively.

5. The method according to claim 1, wherein the lectin gene having the SEQ ID No. 11 is used as reference gene.

6. The method according to claim 1, wherein the nucleic acid sequence SEQ ID NO. 5 or variants thereof obtained by deletion, substitution or addition and having at least 80% homology are used as probe S3.

7. The method according to claim 1, wherein the NA sequence SEQ ID NO. 6 or SEQ ID NO. 6a and the NA sequence SEQ ID NO. 7 or the variants thereof obtained by deletion, substitution or addition and having at least 80% homology are used as primer P3 and primer P4, respectively.

8. The method according to claim 1, wherein the NA sequence SEQ ID NO. 8 or SEQ ID NO. 8a or variants thereof obtained by deletion, substitution or addition and having at least 80% homology are used as synthetic gene fragment of the internal amplification control for transgene determination (target IAC DNA).

9. The method according to claim 1, wherein the NA sequence SEQ ID NO. 10 or SEQ ID NO. 10a or variants thereof obtained by deletion, substitution or addition and having at least 80% homology are used as synthetic gene fragment of the internal amplification control for reference gene determination (reference IAC DNA).

10. The method according to claim 1, wherein the Bt-176 maize gene is detected as transgene.

11. The method according to claim 1, wherein the sequence SEQ ID NO. 12 or variants thereof obtained by deletion, substitution or addition and having at least 80% homology are used as probe S1.

12. The method according to claim 1, wherein the sequence SEQ ID NO. 13 and the sequence SEQ ID NO. 14 or variants thereof obtained by deletion, substitution or addition and having at least 80% homology are used as primer P1 and primer P2, respectively.

13. The method according to claim 1, wherein the invertase gene of maize is used as reference gene.

14. The method according to claim 1, wherein the sequence SEQ ID NO. 15 or variants thereof obtained by deletion, substitution or addition and having at least 80% homology are used as probe S3.

15. The method according to claim 1, wherein that the sequence SEQ ID NO. 16 and the sequence SEQ ID NO. 17 or variants thereof obtained by deletion, substitution or addition and having at least 80% homology are used as primer P3 and primer P4, respectively.

16. The method according to claim 1, wherein the sequence SEQ ID NO. 18 or variants thereof obtained by deletion, substitution or addition and having at least 80% homology are used as target IAC DNA.

17. The method according to claim 1, wherein the sequence SEQ ID NO. 19 or variants thereof obtained by deletion, substitution or addition and having at least 80% homology are used as reference IAC DNA.

18. The method according to claim 1, wherein the nucleic acid sequence SEQ ID NO. 9 or variants thereof obtained by deletion, substitution or addition and having at least 80% homology are used as probe S2.

19. The method according to claim 18, wherein the probe S2 at its 5′ end or its 3′ end is labelled with a reporter fluorescent dye, preferably a fluorescein derivative selected from 6-carboxyfluorescein, tetrachloro-6-carboxyfluorescein, 2,7-dimethoxy-4,5-dichloro-6-carboxyfluorescein, hexachloro-6-carboxyfluorescein, which dye is different from that of probes S1 and S3, and at its other sequence end, the probe S2 is labelled with a quencher, preferably a rhodamine derivative identical to that of probes S1 and S3, preferably with TAMRA.

20. The method according to claim 1, wherein the probes S1 and S3 are labelled at their 5′ end or at their 3′ end with a reporter fluorescent dye, preferably a fluorescein derivative selected from 6-carboxyfluorescein, tetrachloro-6-carboxyfluorescein, 2,7-dimethoxy-4,5-dichloro-6-carboxyfluorescein, hexachloro-6-carboxyfluorescein, preferably with 6-carboxyfluorescein (FAM), and at each of their other ends with a quencher, preferably a rhodamine derivative, preferably with 6-carboxytetramethylrhodamine (TAMRA).

21. A test kit for the quantitative detection of genetically modified DNA (transgene) in foods using fluorescence-coupled PCR, comprising a transgene-specific, fluorescence-labelled probe S1 and two transgene-specific primers P1 and P2, a reference gene-specific, fluorescence-labelled probe S3, and two reference gene-specific primers P3 and P4, as well as a fluorescence-labelled probe S2 as internal amplification control, which probe differs from probes S1 and S3 both in its sequence and its fluorescence-labelling, a synthetic gene fragment (target IAC DNA) having two binding sites for the primers P1 and P2 and one binding site for the probe S2, and a synthetic gene fragment (reference IAC DNA) having two binding sites for the primers P3 and P4 and one binding site for the probe S2.

22. The test kit according to claim 21, used in the detection of the RRS gene.

23. The test kit according to claim 21, used in the detection of the Bt-176 maize gene.

24. The test kit according to claim 21, wherein the kit includes as probe S2 the sequence SEQ ID NO. 9 or variants thereof having at least 80% homology.


SEQUENCE LISTING (25 sequences)

SEQ ID NO: 1 - DNA, 240 bp, Artificial
  Description of Artificial Sequence Roundup soy gene (RRS gene)
gtcttcaaagcaagtggattgatgtgatatctccactgacgtaagggatgacgcacaatcccactatccttcgcaagacccttcctctatataaggaagttcatttcatttggagaggacacgctgacaagctgactctagcagatctttcaagaatggcacaaattaacaacatggctcaagggatacaaacccttaatcccaattccaatttccataaaccccaagttcctaaatctt

SEQ ID NO: 2 - DNA, 24 bp, Artificial
  Description of Artificial Sequence probe
caagctgactctagcagatctttc

SEQ ID NO: 3 - DNA, 21 bp, Artificial
  Description of Artificial Sequence primer
catttggagaggacacgctga

SEQ ID NO: 4 - DNA, 23 bp, Artificial
  Description of Artificial Sequence primer
gggtttgtatcccttgagccatg

SEQ ID NO: 5 - DNA, 24 bp, Artificial
  Description of Artificial Sequence probe
cttcaccttctatgcccctgacac

SEQ ID NO: 6 - DNA, 22 bp, Artificial
  Description of Artificial Sequence primer
gccctctactccacccccatcc

SEQ ID NO: 7 - DNA, 23 bp, Artificial
  Description of Artificial Sequence primer
gaaggcaagcccatctgcaagcc

SEQ ID NO: 8 - DNA, 142 bp, Artificial
  Description of Artificial Sequence target IAC DNA for the RRS gen
catttggagaggacacgctgaggacgttcgccaattttcgcctcccacgtctcaccgagcgtggtgtttacgaaggttttacgttttcccgtatcccctttcgttttcatccagtctttcatggctcaagggatacaaaccc

SEQ ID NO: 9 - DNA, 21 bp, Artificial
  Description of Artificial Sequence probe
tcgcctcccacgtctcaccga

SEQ ID NO: 10 - DNA, 150 bp, Artificial
  Description of Artificial Sequence reference IAC DNA for the RRS gen
gccctctactccacccccatccggacgttcgccaattttcgcctcccacgtctcaccgagcgtggtgtttacgaaggttttacgttttcccgtatcccctttcgttttcatccagtctttgacaatcggcttgcagatgggcttgccttc

SEQ ID NO: 11 - DNA, 250 bp, Artificial
  Description of Artificial Sequence lectin gene
gggaaagttacaactcaataaggttgacgaaaacggcaccccaaaaccctcgtctcttggtcgcgccctctactccacccccatccacatttgggacaaagaaaccggtagcgttgccagcttcgccgcttccttcaacttcaccttctatgcccctgacacaaaaaggcttgcagatgggcttgccttctttctcgcaccaattgacactaagccacaaacacatgcaggttatcttggtcttttcaac

SEQ ID NO: 12 - DNA, 21 bp, Artificial
  Description of Artificial Sequence probe
atgtccaccaggcccagcacg

SEQ ID NO: 13 - DNA, 21 bp, Artificial
  Description of Artificial Sequence primer
cccatcgacatcagcctgagc

SEQ ID NO: 14 - DNA, 20 bp, Artificial
  Description of Artificial Sequence primer
caggaaggcgtcccactggc

SEQ ID NO: 15 - DNA, 23 bp, Artificial
  Description of Artificial Sequence probe
ccacctcttctaccagtggaacc

SEQ ID NO: 16 - DNA, 21 bp, Artificial
  Description of Artificial Sequence primer
cgctgtatcacaagggctggt

SEQ ID NO: 17 - DNA, 20 bp, Artificial
  Description of Artificial Sequence primer
agcggtaggtgcagccagtg

SEQ ID NO: 18 - DNA, 136 bp, Artificial
  Description of Artificial Sequence target IAC DNA for the Bt-176 maize gen
cccatcgacatcagcctgagcgttcgccaattttcgcctcccacgtctcaccgagcgtggtgtttacgaaggttttacgttttcccgtatcccctttcgttttcatccagtcttttgccagtgggacgccttcctg

SEQ ID NO: 19 - DNA, 136 bp, Artificial
  Description of Artificial Sequence reference IAC DNA for the Bt-176 maize gen
cgctgtatcacaagggctggtgttcgccaattttcgcctcccacgtctcaccgagcgtggtgtttacgaaggttttacgttttcccgtatcccctttcgttttcatccagtcttttcactggctgcacctaccgct

SEQ ID NO: 20 - DNA, 24 bp, Artificial
  Description of Artificial Sequence probe
cccactatccttcgcaagaccctt

SEQ ID NO: 21 - DNA, 21 bp, Artificial
  Description of Artificial Sequence primer
ctgacgtaagggatgacgcac

SEQ ID NO: 22 - DNA, 24 bp, Artificial
  Description of Artificial Sequence primer
aagatctgctagagtcagcttgtc

SEQ ID NO: 23 - DNA, 21 bp, Artificial
  Description of Artificial Sequence primer
cgttgccagcttcgccgcttc

SEQ ID NO: 24 - DNA, 143 bp, Artificial
  Description of Artificial Sequence target IAC DNA for the RRS gen
ctgacgtaagggatgacgcacggacgttcgccaattttcgcctcccacgtctcaccgagcgtggtgtttacgaaggttttacgttttcccgtatcccctttcgttttcatccagtctttgacaagctgactctagcagatctt

SEQ ID NO: 25 - DNA, 149 bp, Artificial
  Description of Artificial Sequence reference IAC DNA for the RRS gen
cgttgccagcttcgccgcttcggacgttcgccaattttcgcctcccacgtctcaccgagcgtggtgtttacgaaggttttacgttttcccgtatcccctttcgttttcatccagtctttgacaatcggcttgcagatgggcttgccttc